(19) United States
(12) Patent Application Publication
(10) Pub. No.: US 2023/0277915 A1
(43) Pub. Date: Sep. 7, 2023

(54) Stackable 3D Tic Tac Toe

(71) Applicant: Carlton Richard McCracken, JR., Curwensville, PA (US)

(72) Inventor: Carlton Richard McCracken, JR., Curwensville, PA (US)

(21) Appl. No.: 18/078,117

(22) Filed: Dec. 9, 2022

Related U.S. Application Data
(60) Provisional application No. 63/299,970, filed on Jan. 15, 2022.

(51) Int. Cl. A63F 3/00; G16H 20/00

(52) U.S. Cl. CPC A63F 3/00094; A63F 3/00214; A63F 3/00697; G16H 20/00; A63F 2003/0022; A63F 2003/00716

(57) ABSTRACT

The challenge to myself was to create a game that would negate my three dislikes in playing the “age old” Tick-Tac-Toe game.
#1 dislike: The Tick-Tac-Toe game usually ends in a tie/stalemate, boring!
#2 dislike: Whoever goes first in the Tick-Tac-Toe game, controls/dominates the game, being a bully.
#3 dislike: The Tick-Tac-Toe game comes to an end too quickly without a chance to use mental extended strategy, not challenging enough.

“Bottom Line:” in Stackable-3D-Tick-Tac-Toe, there is always a winner, Stackable-3D-Tick-Tac-Toe is a “winner” of a game and a helpful, healing Therapy Board!

Endorsement: Richard McCracken has developed an awesome “Stackable 3D Tic Tac Toe Board” which can be utilized as a vertical game board or as a functional activity. This board game is unlike anything I have seen in 20 years practicing as an occupational therapist.

I utilize this game daily to promote fine motor coordination, strength, and pinch strength.

### CROSS-REFERENCE TO RELATED APPLICATIONS

Provisional application submitted Jan. 15, 2022, EFS ID: 44756781, Application No. 63/299,970, Confirmation Number: 7466, First Named Inventor: Carlton Richard McCracken Jr., Receipt Date: 15 Jan. 2022, Time Stamp: 21:32:58, Application Type: Provisional

### STATEMENT REGARDING FEDERALLY SPONSORED RESEARCH OR DEVELOPMENT

(Not Applicable)

I used my own money for research and development, no federal money was used.

### REFERENCE TO A “SEQUENCE LISTING”

(Not Applicable)

In this application, there is no need to include large sets of information on compact dis(s)

### BACKGROUND OF THE INVENTION

On 12/20/20 around 18:00, I was in my old, small, cold, junky wood shop trying to make a small wooden toy trailer for a home-made Christmas present for my youngest grandson. I was using my table saw that I have used for around 40 years without accident, but such was not the case at this particular time. I cut three of my fingers on my left hand on the saw blade but with a trip to the ER and emergency surgery the next day, praising God, my fingers were saved! So, then it was off to Occupation Therapy, OT, for a couple months. At OT, one of the therapy exercises was that I was given a block of wood that had random location of bolts running through the block of wood and then fasted with nuts. The bolt heads were countersunk but the bolts spun in the oversized holes. As I started the finger therapy with this block of wood and combination of bolts and nuts, I knew I could and needed to make a much better block of wood with bolts and nuts added for therapy of the wrist, hand, and fingers. I explained to Kelly, the OT specialist, that I would make for her a much better “therapy board” for her OT department. I told her I would make a “therapy board” that would be customized as a Tick-Tac-Toe game with carriage bolts which would not spin but stay in place as nuts were screwed on. To have the teams differentiated, the one player would use regular nuts and the other player would use wingnuts. Kelly was very excited about getting a “therapy board” that would be a game as well, helping us patients to better focus and enjoy the therapy task at hand, (yes pun intended). Kelly explained the color she would like for the “therapy board”. So, I collected my thoughts and produced a Tick Tack Toe game as a therapy board.

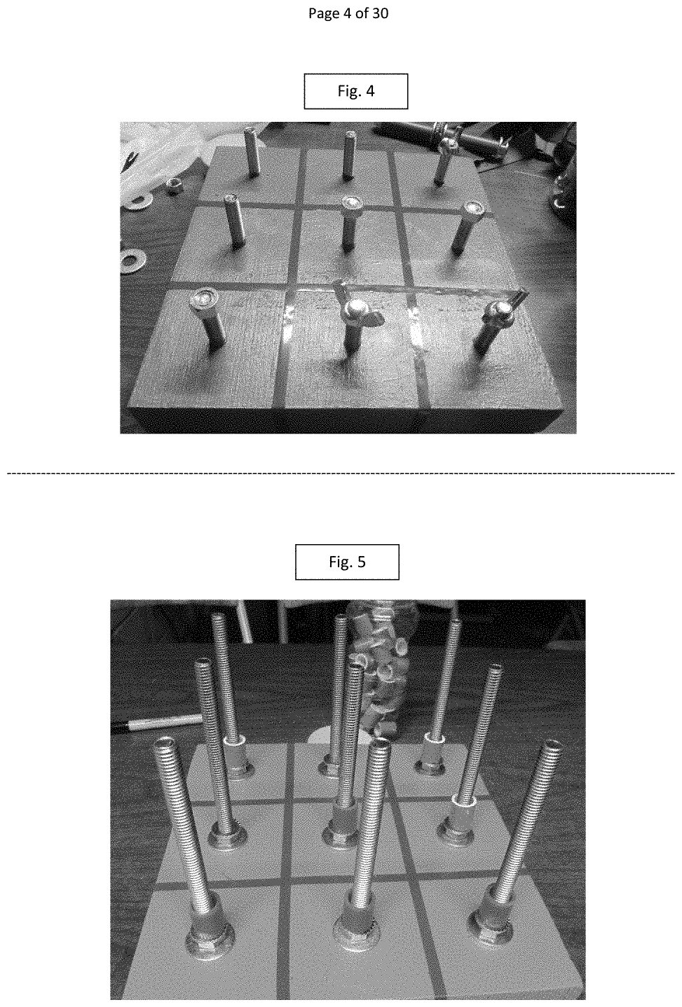

FIG. 4, in the Drawings File, page 4, shows the first attempt of producing just a basic Tic Tac Toe game as a therapy board.

Kelly was thrilled with the finished product and how it would be such a help to her patients, which included me. Kelly encouraged me, even at this point, to get a patent on this therapy board. She explained that she had never seen such a therapy board in all her years searching through OT and PT therapy exercise catalogs. Kelly also explained that if there was such a similar therapy board, she believed that it would cost over $200.

But just as with the first therapy board I used, I knew I could and needed to make a better Tick-Tac-Toe game as a therapy board. The challenge to myself was to create a game that would negate my three dislikes in playing the “age old” regular Tick-Tac-Toe game.
#1 dislike: The Tick-Tac-Toe game usually ends in a tie/stalemate, boring!
#2 dislike: Whoever goes first in the Tick-Tac-Toe game, controls/dominates the game, being a bully.
#3 dislike: The Tick-Tac-Toe game comes to an end too quickly without a chance to use mental extended strategy and the physical motor skills of the wrist, hand, and fingers, not challenging enough.

### BRIEF SUMMARY OF THE INVENTION

This challenge led to the development of the game I call:

### “Stackable-3D-Tick-Tac-Toe, Dexterity, Determination, and Depth”, For Game or Rehab Therapy of Injured Fingers and Stroke Victims or Play the Game for Therapy!

### The Rules of the Game/Therapy Board

[Regular Play—2 Players] or [Partner Play—4 Players, 2 players (Partners/Team—Red game pieces) and 2 players (Partners/Team—Blue game pieces), same rules just playing as partners.]

Rules: There is a winner every time! One wins by meeting the actions listed in either of the rules, 1, 2, or 3:
1. Horizontally/Across—having 3 of your color pieces in a row on the same level, (regular Tick-Tac-Toe per level).
2. Vertically/Upward—having 4 of your color pieces touching each other.
3. If a player knocks over any stack of pieces, that player loses.
4. The player that goes first cannot place one of his pieces vertically/upward until the player that goes second, does so. When the first game piece is placed on the second level, the second level does not need to be filled by any number of game pieces as the next player could place his game piece in the third level on top of the piece that was just placed in the second level. This pattern of play continues for all levels until there is a win.

Summary: Play continues both horizontally/across and vertically/upward until one of the players wins or loses as described in Rules 1, 2, or 3. There is always a winner of every game! If the game is being played for rehab therapy, the opponent of the injured player, needs to play with lest dominate hand for fairness. Patent Pending!

### BRIEF DESCRIPTION OF DRAWINGS

There is a separate file, named “Drawings”, that contains three black and white, fundamental CAD drawings of some of the different versions of the game/therapy board, Stackable 3D Tic Tac Toe. Also, in the separate Drawing file are approximately 50 figures that help illustrate the text in the Specification file. I have made a request for the acceptance of these 50 figures by the USPTO Reviewers by using a “Self-Made Form” to Petition The USPTO Reviewers to Accept the Colored Screen Shots and Photos. This completed “Self-Made Form” is being sent along with the Corrected Specification file and the Corrected Drawing file.

### DETAILED DESCRIPTION OF THE INVENTION

Following is text describing the three main CAD drawings that will be listed multiple times throughout this non-provisional utility application. All three of these CAD drawings are listed in the separate “Drawings File” of this application.

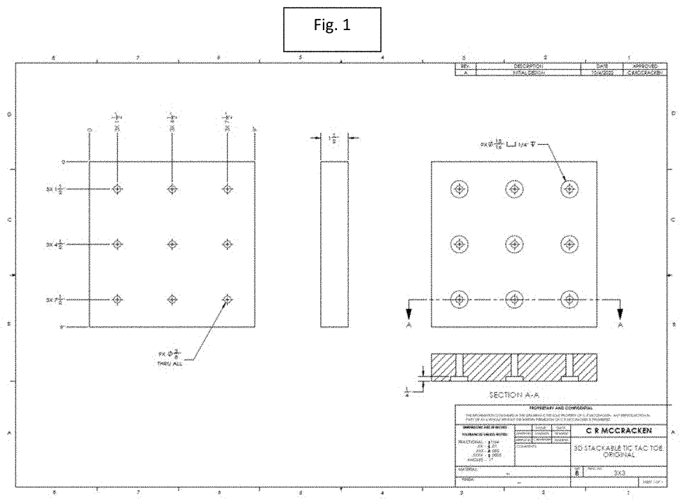

Shown in FIG. 1, located in the Drawings file, page 1, is the original design and size, 9 inch by 9 inch, square, 3 bolts by 3 bolts=total of 9 bolts,

The following text along with Figures/Pictures shown in the Drawings file helps to visualize the rules.

FIG. 5, located in the Drawings file, page 4 shows the player with the red pieces has won with 3 red pieces in a row, horizontally on the first level as in regular Tick-Tac-Toe.

FIG. 6, located in the Drawings file, page 5 shows the player with the red pieces has won with 3 red pieces in a row, horizontally on the 2nd level.

FIG. 6 is an example of Rule (1) where to win, the 3 red pieces must be in the same horizontal level.

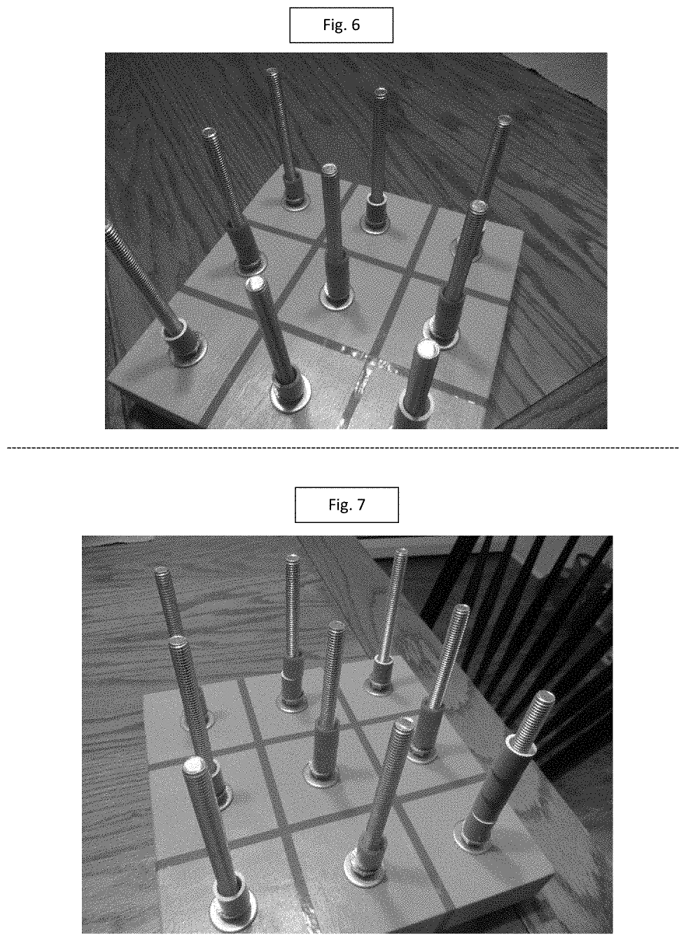

FIG. 7, located in the Drawings file, page 5, shows the player with the blue pieces has won with all 4 blue pieces touching vertically. FIG. 7 is an example of Rule (2) where to win, Vertically/Upward—having 4 of your color pieces touching each other.

FIG. 8, located in the Drawings file, page 6, is an “almost” example of Rule (3) where to win, you or your opponent, in the act of stacking, knocks over any stack of pieces, loses the game. Do not forget that as you and your opponent place pieces higher and higher vertically, a win also can be accomplished with 3 pieces in a row, horizontally on the same level or 4 pieces of same color touching vertically.

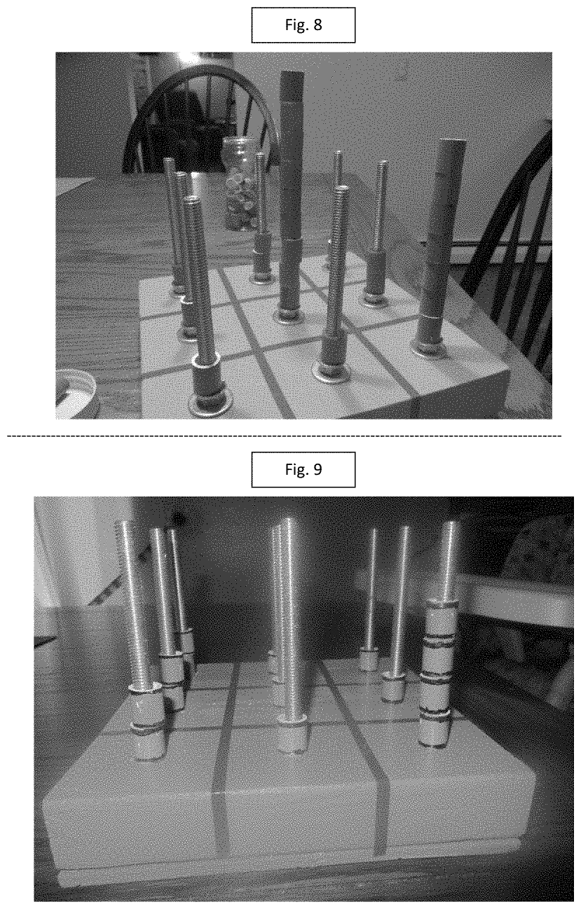

FIG. 9, located in the Drawings file, page 6, shows that the game pieces can be marked with a permanent marker all around each end which greatly helps distinguish the levels. Marking the pieces in this way takes a very long time! Many of the players like the pieces unmarked so they can more easily “sneak” in a win.

Review and Explanation of the Injury Therapy Side: Kelly, the OT specialist, was even more excited when presented with the advanced and updated game of “Stackable-3D-Tick-Tac-Toe, Dexterity, Determination, and Depth”! Much of the time, Kelly would not use the new therapy board as a game as getting patients into pairs to play each other was difficult. Now with the greater height/“Depth” of the bolts, the patients of which I was one, could build “Dexterity” if they were “Determined” to do so! The last sentence shows the three words in bold and underlined that I wanted to be used in the subtitle. The “3D” in the main title, of course refers to the playing of the game going both horizontally and vertically which associates with the “Depth” in the subtitle. The “3D” also represents the other two “D” words of “Dexterity” and “Determined” which I saw in the rehabilitation of my follow patients. It was very encouraging and satisfying to see my follow patients using my creation of a therapy board to help themselves, rehabilitate from their stroke, wrist, hand, and finger injuries! Kelly, the OT and PT specialist, added a large tweezer to the therapy board setup. The tweezer was used instead of fingers at times, to squeeze/pinch the game pieces to both “add to” and “take off” from the bolts. Using the tweezer used muscles and developed muscles in a different way than using solely the fingers to “add to” and “take off” the game pieces from the bolts.

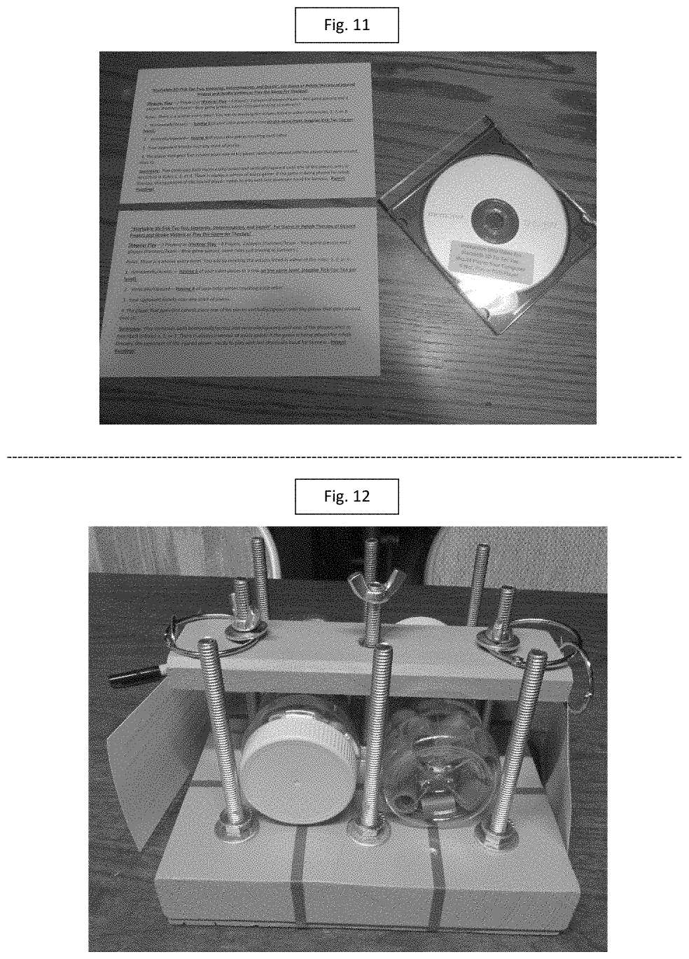

FIG. 12, located in the Drawings file, page 8, shows there are also wingnuts used in this updated version to hold the handle down on the two containers of game pieces.

These wingnuts are also very valuable in being used with the therapy board. With the wingnuts being screwed/twisted onto the bolts in using the injured hand or wrist or fingers, rehabilitation is accomplished for the muscles and tendons.

Following is a letter from Kelly who was my occupational therapist which explains how she uses “Stackable 3D Tic Tac Toe” as both a therapy board and a game. The letter was converted from a pdf file so there are a couple of small conversion issues that can be seen.

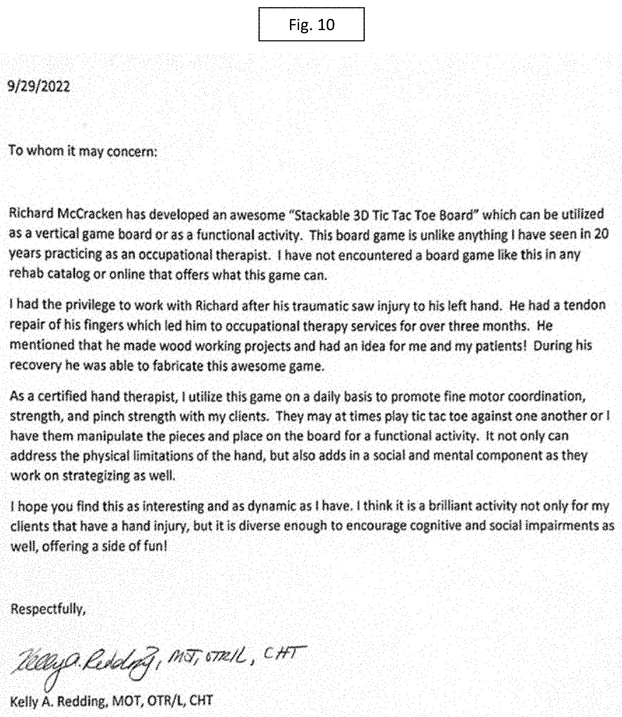

FIG. 10, located in the Drawings file, page 7 shows an exact copy of the therapy endorsement letter, showing Kelly's signature.

The letter is not intended to be “bragging” about the game/therapy board but to help recognize the functionality of the game/therapy board and how it achieves its intended and sometimes non-intended purposes and goals.

Sep. 29, 2022

To whom it may concern:

Richard McCracken has developed an awesome “Stackable 3D Tic Tac Toe Board” which can be utilized as a vertical game board or as a functional activity. This board game is unlike anything I have seen in 20 years practicing as an occupational therapist. I have not encountered a board game like this in any rehab catalog or online that offers what this game can.

I had the privilege to work with Richard after his traumatic saw injury to his left hand. He had a tendon repair of his fingers which led him to occupational therapy services for over three months. He mentioned that he made wood working projects and had an idea for me and my patients! During his recovery he was able to fabricate this awesome game.

As a certified hand therapist, I utilize this game on a daily basis to promote fine motor coordination, strength, and pinch strength with my clients. They may at times play tic tac toe against one another or I have them manipulate the pieces and place on the board for a functional activity. It not only can address the physical limitations of the hand, but also adds in a social and mental component as they work on strategizing as well.

I hope you find this as interesting and as dynamic as I have. I think it is a brilliant activity not only for my clients that have a hand injury, but it is diverse enough to encourage cognitive and social impairments as well, offering a side of fun!

Respectfully,

Kelly A. Redding, MOT, OTR/L, CHT

Review and Explanation of the Game Side: With the advanced and updated game of “Stackable-3D-Tick-Tac-Toe, Dexterity, Determination, and Depth” the playing of the game has been tested by many members of my family and friends and myself and have found it to be a lot of fun to play. The game is easy to play with just four basic rules to follow. The game comes with two copies of the rules and a game summary on brightly colored cardstock along with a DVD containing a game instructional video. There are two of these cards per game so that each player can have a rule card to use for review and strategy.

FIG. 11, located in the Drawings file, page 8 shows that two rule cards along with an instructional DVD are included with the game/therapy board.

FIG. 12, located in the Drawings file, page 8, Shows the rule cards used to be on 4×6 picture paper hanging from two of the ends of the game therapy board.

As the rules expanded, the 4×6 size had to be expanded as well. The design of how the game/therapy board is packaged and transported has been updated but the original still has some advantages.

FIG. 12, located in the Drawings file, page 8, shows the original setup is balanced well and with either using the handle or the center elevated wingnut, the unit can be transported easily without concern of losing parts and pieces.

FIG. 13, located in the Drawings file, page 9, shows the new packaging is a simple but well-made backpack with large side zippered pockets to hold the two rule cards and DVD.

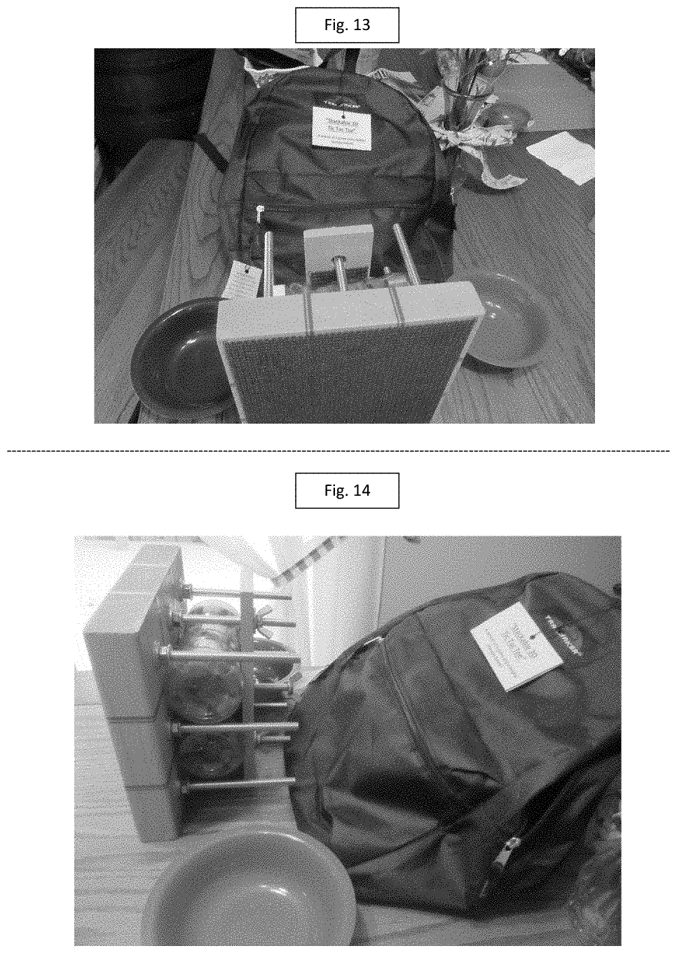

FIG. 14, located in the Drawings file, page 9 shows the orientation of the game/therapy board to be placed into the backpack so the handle can act as a “rest”, so it can sit upright in the backpack.

Another occupational therapist explained that with the game/therapy board on its side, this is another way that stroke victims can use the board, so they are placing the pieces on the bolts at a different angle. The backpack can hold bowls to hold game pieces and also an additional color container, white game pieces, so a third player can be included, red, blue, and now white. Having three players really challenges one's mind but it works as it has been tested and it is a lot of fun with a different type of slant!

The original game can also be played involving four players divided into two teams. [Partner Play—4 Players, 2 players (Partners/Team—Red game pieces) and 2 players (Partners/Team—Blue game pieces), same rules just playing as partners.]

FIG. 15, located in the Drawings file, page 10 shows that I then designed the “Stackable 3D Tic Tac Toe Mega Board, Square, 5×5”.

The mega board brings increase challenges with additional players with additional color pieces and additional playing area. Moving from the traditional 3×3 grid of Tic Tac Toe to the Mega Board, “blows one's mind”! If there are only two players using the mega board, the rules must be changed. All these “mind blowing” challenges and additions will be sorted out and details given, in the following “claims Section”.

The game is usually played at a fast pace at the beginning but as the variables of opportunities of moves increase, the players slow down watching cautiously in how to move to win or block. This game holds your interest and focus as each move is so important as whether you will win or lose. This game is great for tournament play with many options such as a player can advance by winning two out of three games and then advancing on to play in the next level of the tournament. The size of all the parts of Stackable-3D-Tick-Tac-Toe can be reduced and enlarged in size and materials to meet the desires of play but the present or “default” size and use of materials, have been proved not only acceptable but possibly be the “best fit” for common game enjoyment! The entire bottom of the game board is glued and covered with one piece of non-slide and gripping shelve liner material which gives a great advantage of cushioning and a non-marring effect. In unpacking the unit to play the game and then packing the unit back up after game is a “neat”/enjoyable game in itself of “puzzle connecting assembly”! “Bottom Line:” in Stackable-3D-Tick-Tac-Toe, there is always a winner, Stackable-3D-Tick-Tac-Toe is a “winner” of a game!

The dimensions of the game, the board, the pieces, and the accessories will be listed as to what was used but really any size within limitations can be used and still be functional in the game being played, could be used. Not only can the dimensions be viable but also the materials as well. The carriage bolts could be replaced with wood dowel rods, plastic studs, etc. The wooden base could be metal or plastic, etc. With this thought in mind, I will list the size and materials used and then also list the phrase “or equivalent” or “preferably”. I can see this game going to market in many ways, but I would think the two main avenues would be a rugged, heavy duty version as the one being described and another version that is much cheaper, much lighter, and made of plastic in cardboard game box. Other visionary game/therapy board possible versions are listed towards the end of this Specification file and in the claims section.

The game board is a regular 2 by 10 purchased from Lowes or other lumber yard and cut to make a square, (or equivalent). A finished 2 by 10 is around 9⅛ inch wide. So, the 2 by 10 would be cut into a square with the length of the sides being 9 inches. The red, white, orange, and blue, (or equivalent) game pieces which I am calling “Tubies” as they are cut from plumbing tubes and the “Tubies” name was suggested by my friend Mark. These “Tubies” are either cut from plumbing pipe of ½ PVC or PEX tubing, (or equivalent), ¾ inch long, (or equivalent).

FIG. 9, located in the Drawings file, page 6 shows that the PVC tubing, the blue and green pieces, cuts the “cleanest”/straightest and is the easiest to stack as the PVC is harder than the PEX tubing.

You can purchase from Lowes the PEX tubing in 10 foot sticks in bule, red, white, and orange, (orange PEX is twice the cost as it is made for heating system). The PVC tubing can be purchased at Lowes or local hardware stores as it is more common.

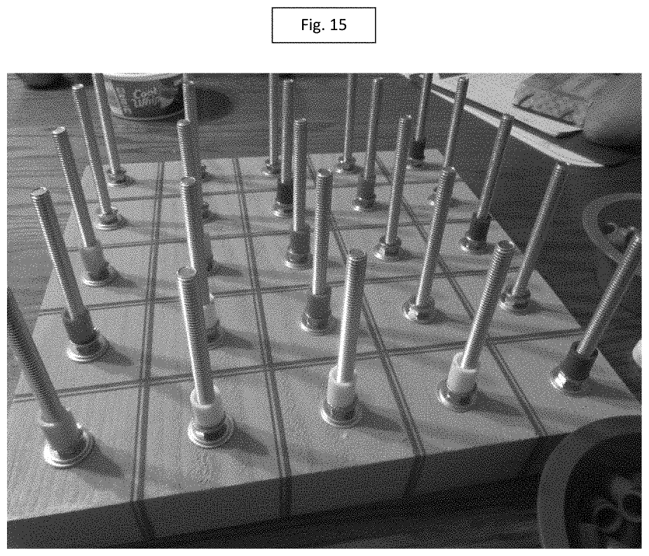

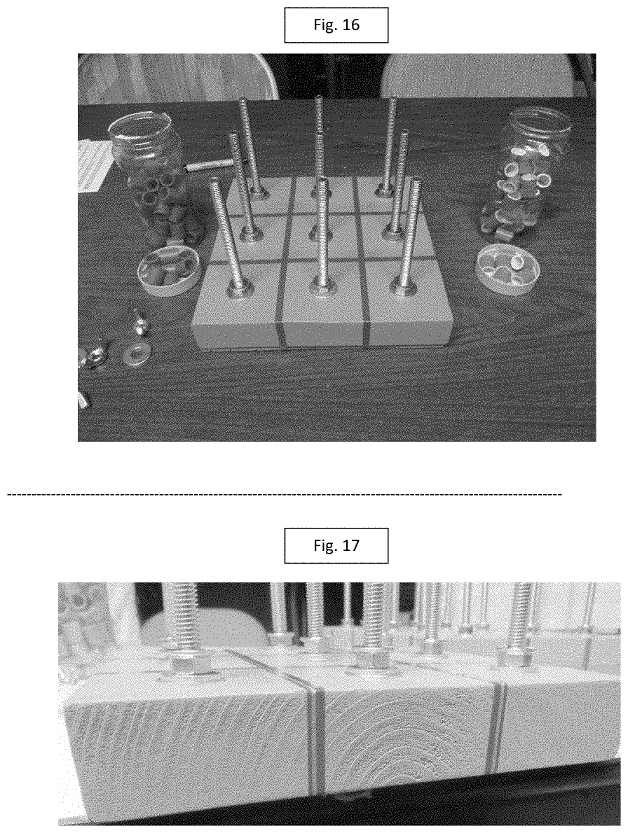

FIG. 16, located in the Drawings file, page 11 shows that I had to paint the PVC tubing different colors, such as blue, as the PVC tubing only comes in a tan color.

You can buy the PVC tubing, special order, in different colors, but it is very costly for the product and the shipping. I found the best, easiest, and safest way for me to cut the tubing was with a handheld tubing cutter.

The long pegs/bolts are ⅜, 6 inch long carriage bolts, (or equivalent), needing 9 of them.

In starting to mark out the bolt holes, the first important decision to be made, is which side to use as the bottom when using a wood base. There are two factors to consider, the priority is to choose the bottom side that will sit/lay without “wobbling”.

FIG. 17, located in the Drawings file, page 11 shows that wood tends to wrap with the direction of the end grain.

The middle of the board “heaves up”/“arches up” just as the end grain does. This leaves the outside edges of the board to be in contact with the table which produces stability with the “no wobble” effect. Really, this is the main factor in choosing which side will be the bottom and one hopes the thus determined bottom side, will also be the worst looking as it will be covered with an antiskid shelf liner material.

There are two ways that I know of and have tried to lay out the Tick-Tac-Toe grid. The first one that I used, and it worked well, is to divide the square into 3 equal columns both ways. With this square of 9 inches, marks would be made along all 4 edges at 3 and 6 inches.

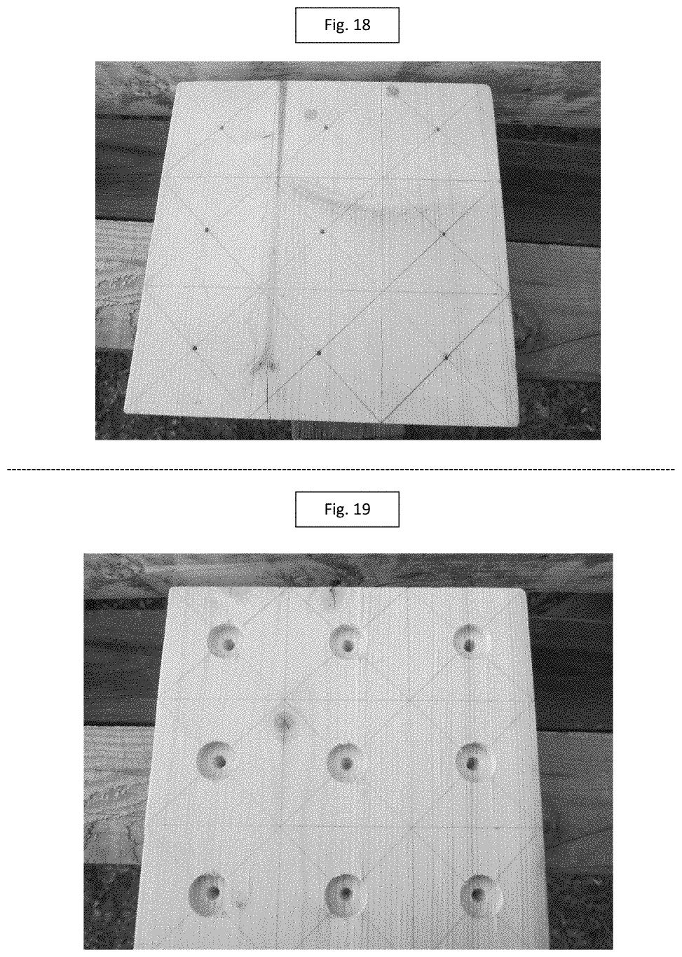

FIG. 18, located in the Drawings file, page 12, shows that once the 3 columns are marked out both ways on the game board, diagonal lines are drawn through all 9 squares, both ways, to establish the center of each of the 9 squares.

The second way of marking out the grid, is where the bolt holes will be drilled directly, would be to measure and mark from two sides that are perpendicular to each other, three lines, marked at 1.5, 4.5, and 7.5 inches using a marking square. Where the three lines of both sides intersect, is where the bolt holes need to be drilled.

FIG. 19, located in the Drawings file, page 12 shows that the center of each of the 9 squares are “center punched” and drilled with a 15/16 inch spade bit to a depth of just over a ¼ inch to “counter sink” the heads of the carriage bolts.

FIG. 19, located in the Drawings file, page 12 also shows that once the “counter sinking” is completed for all 9 squares, a ⅜ spade bit is used to drill down through the center of each of the “counter sinks”, clear through the game board.

I then use a counter-sink bit to ream the tops of the ⅜ inch holes to prevent as best as possible, the wood around the top of the hole from splintering when the carriage bolts are pushed or hammered through the drilled holes.

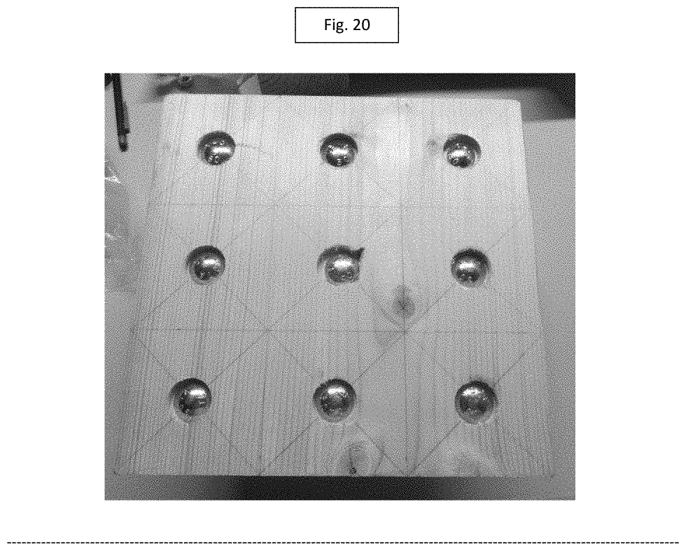

FIG. 20, located in the Drawings file, page 13, shows that the 6 inch carriage bolts are hammered through the ⅜ inch holes and seated with several raps from a smaller hammer on the heads of the carriage bolts.

The game board is flipped over, and ⅜ flat washers and then 7/16 flat washers are placed on the bolts. Then ⅜ serrated lock nuts are placed upside down and tightened down on all 9 carriage bolts to establish both a perpendicular fit and a tight support of the carriage bolt to the game board.

FIG. 8, located in the Drawings file, page 6 shows the tightened serrated lock nuts produce a great small platform for the game pieces to be stacked on.

The game board top and all 4 sides are painted orange, (or equivalent), and left to dry.

FIG. 21, located in the Drawings file, page 14 shows that red, ¼ inch self-adhesive Pinstripe for detailing vehicles, (or equivalent), is used for outlining the Tick-Tac-Toe grid.

FIG. 21, located in the Drawings file, page 14 also contains both a picture of the game/therapy board and also a table listing detailed descriptions and dimensions of many of the parts.

The containers that hold the 50 each, the different colors of the game pieces, are purchased at my local Dollar General Store as they hold Dry Roasted peanuts, net wt. 7.5 oz.

FIG. 12, located in the Drawings file, page 8 shows that these Dry Roasted peanuts containers were used as they are “see through” and fit well in between the carriage bolts.

The containers are held in place by slight pressure by a wooden handle that spans the length of the game board. This handle is made from approximately ⅜ to ¾ inch thick wood, with three holes that match the middle row of bolts. This allows for ⅜ washers and wingnuts to be screwed down on the two outside bolts and hold the containers in place for secure and easy transport. The handle allows for you to get your fingers around it for transport.

FIG. 12, located in the Drawings file, page 8 also shows that a wingnut that is screwed down just below the tip of the center bolt, makes for a good and balanced additional handle for transport.

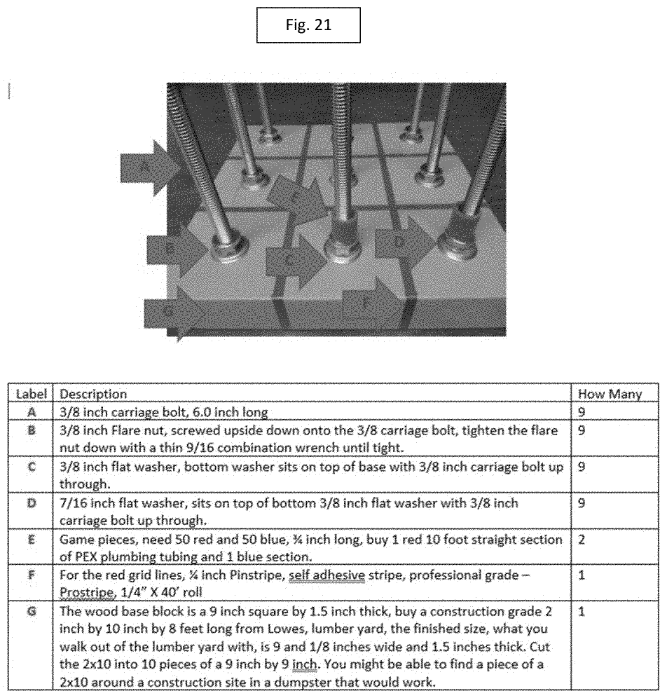

FIG. 22, located in the Drawings file, page 15 shows that the entire bottom of the game board is glued and covered with one piece of non-slide and gripping shelf liner material which also gives a cushioning and non-marring effect.

The glue that I use is “Clear Gorilla Glue” which is strong and what glue that comes out from the bottom material, dries to the same non-skid texture.

FIG. 23, located in the Drawings file, page 16 shows the non-slide and gripping shelf liner material with the brand and details which was purchased at Walmart.

FIG. 9, located in the Drawings file, page 6 shows that in the original prototypes and some of the other figures, there was a ¼ inch piece of plywood that was screwed to the bottom of the game board to hold the carriage bolts from dropping out.

With adding into the production process, the “countersinking” of the bottom of the game board for the carriage bolts heads and with screwing down the serrated lock nuts on top of the game board, there was no need for the ¼ piece of plywood, saving cost and time.

The game is easy to play with just four rules to follow. I believe with the text explanations and the many figures that are displayed in the Drawings file, one would know the dimensions, materials, and supplies needed to build my fun game and therapy board of Stackable-3D-Tick-Tac-Toe.

“In Thinking Outside the Box”/“Visionary Thoughts”:

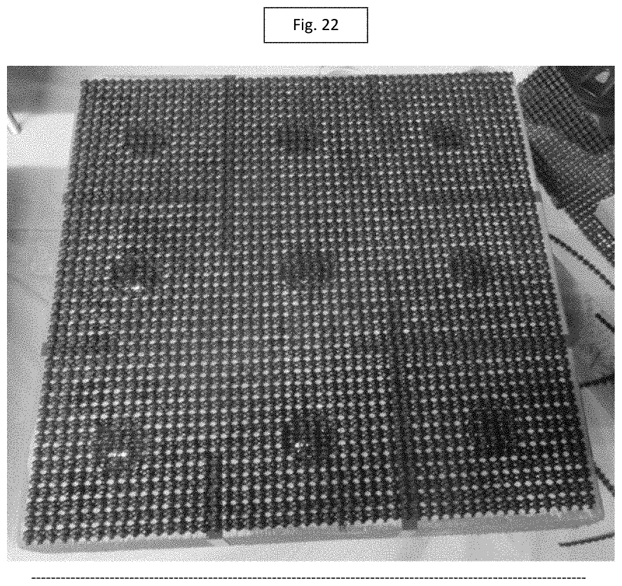

FIG. 24, located in the Drawings file, page 16 shows there can be a grid naming process established so moves can be recorded, games played over the internet or by mail.

I have had some computer game Beeks play this game which they enjoyed very much, in working their strategy. As they played the game and thought, they then wanted to make a digital game version of Stackable-3D-Tick-Tac-Toe. As with other board games that lent themselves to being enlarged to such a point that they were played on a court at a playground or park, such could be the case for Stackable-3D-Tick-Tac-Toe. You could have poles in the ground in a Tick-Tac-Toe grid, the game pieces would be lightweight but large. The two teams could have numerous players but the same number of players on each team. The team players would get a great athletic workout in getting the game pieces up over the poles by using ladders, human ladder chains, or ropes or established ladders at each pole. This could be a great strength training and climbing game for the military and police and could even become a college and Olympic sport! Adding clock timing such as using a clock in chess matches is an option to further add to the strategies of playing Stackable-3D-Tick-Tac-Toe wherever and however it is played.

1. FIG. 1, located in the Drawings file, page 1 shows that claim #1 comprises of the original 9 inch by 9 inch, Square, 3 bolts by 3 bolts=total of 9 bolts of Stackable 3D Tic Tac Toe.
This claim not only claims the 9 inch by 9 inch, 3 bolts by 3 bolts=total of 9 bolts of Stackable 3D Tic Tac Toe, but any size of square game board, either larger or smaller where the game of “Stackable 3D Tic Tac Toe” can functionally be played.
FIG. 12, FIG. 13, and FIG. 21, located in the Drawings file, pages 8, 9 and 14 respectfully shows the items that comprise this version of “Stackable 3D Tic Tac Toe” in this claim.
1. The game/therapy board, handle, and hardware
2. 50 of the red game pieces/tubies in the wide mouth plastic container.
3. 50 of the blue game pieces/tubies in the wide mouth plastic container.
4. A red and a blue plastic bowl for the game pieces/tubies to be poured into for better handling. These bowls are also used to cover the bolts once the board is placed into the backpack to stop any punctures through the backpack.
5. Two rule cards and DVD with instruction video.
6. Backpack for storage and transportation of game/therapy board.
FIG. 25, located in the Drawings file, page 17, and FIG. 12, located in the Drawings file, page 8 shows that the original design of “9 inch by 9 inch, Square, 3 bolts by 3 bolts”, its rules, design, and size dimensions have been a really “good fit” for the game/therapy board.
It has proved to be a solid basis to use to extrapolate to the many other versions and sizes of Stackable 3D Tic Tac Toe!
The Rules of the Game/Therapy Board
[Regular Play—2 Players] or [Partner Play—4 Players, 2 players (Partners/Team—Red game pieces) and 2 players (Partners/Team—Blue game pieces), same rules just playing as partners.]
Rules: There is a winner every time! One wins by meeting the actions listed in either of the rules, 1, 2, or 3:
1. Horizontally/Across—having 3 of your color pieces in a row on the same level, (regular Tick-Tac-Toe per level).
2. Vertically/Upward—having 4 of your color pieces touching each other.
3. If a player knocks over any stack of pieces, that player loses the game.
4. The player that goes first cannot place one of his pieces vertically/upward until the player that goes second does so. When the first game piece is placed on the second level, the second level does not need to be filled by any number of game pieces as the next player could place his game piece in the third level on top of the piece that was just placed in the second level. This pattern of play continues for all levels until there is a win.
Summary: Play continues both horizontally/across and vertically/upward until one of the players wins or loses as described in Rules 1, 2, or 3. There is always a winner of every game! If the game is being played for rehab therapy, the opponent of the injured player needs to play with lest dominate hand for fairness. Patent Pending!

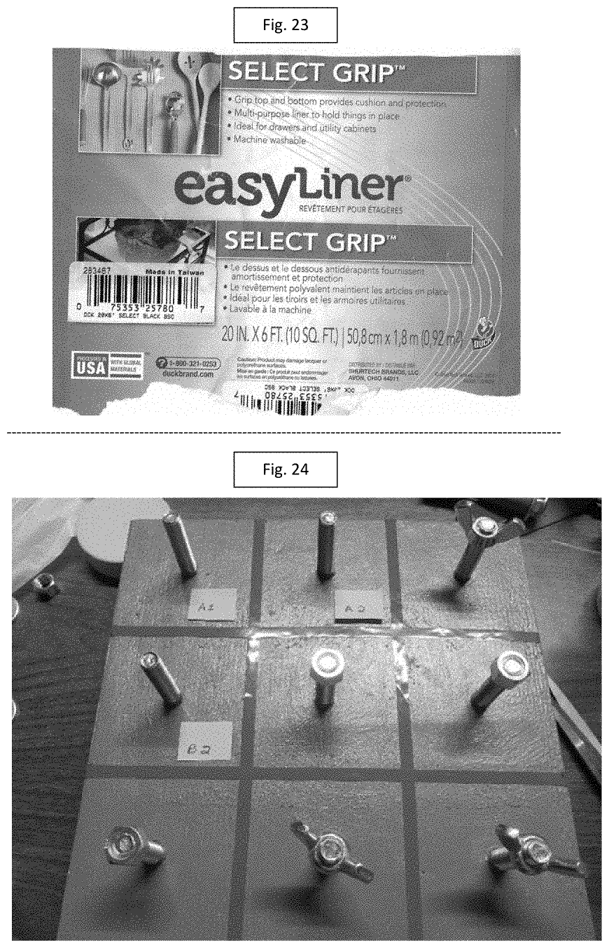

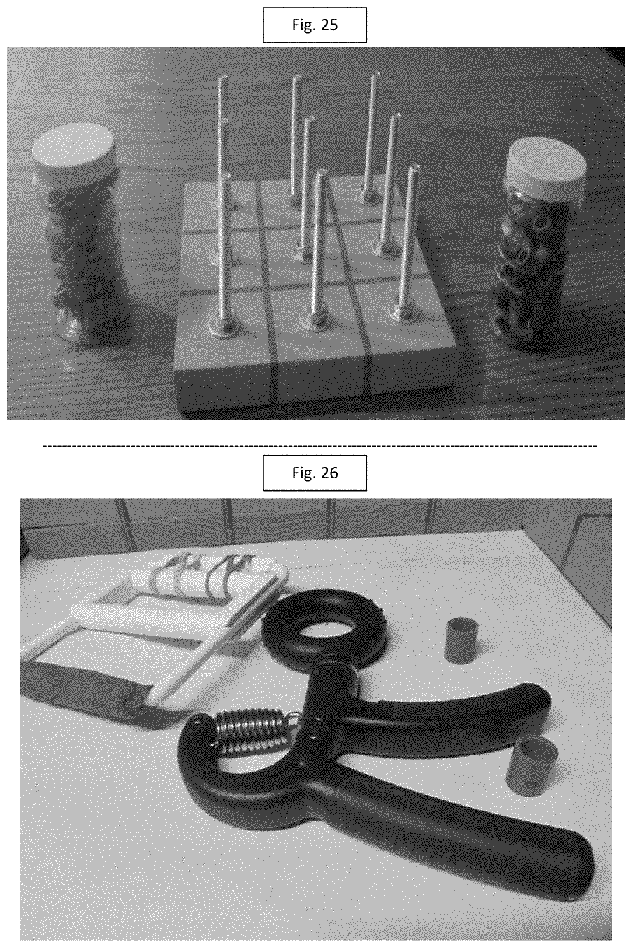

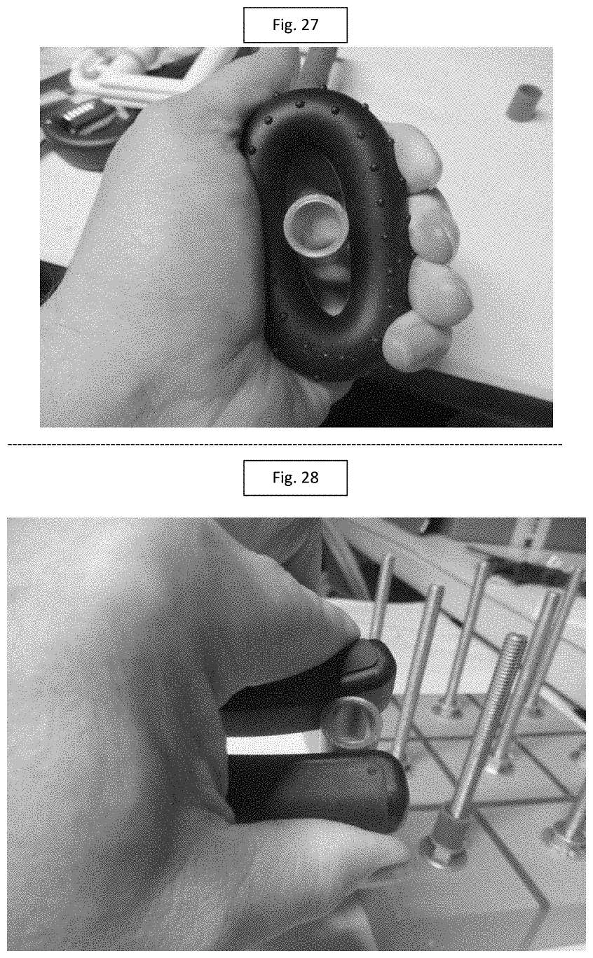

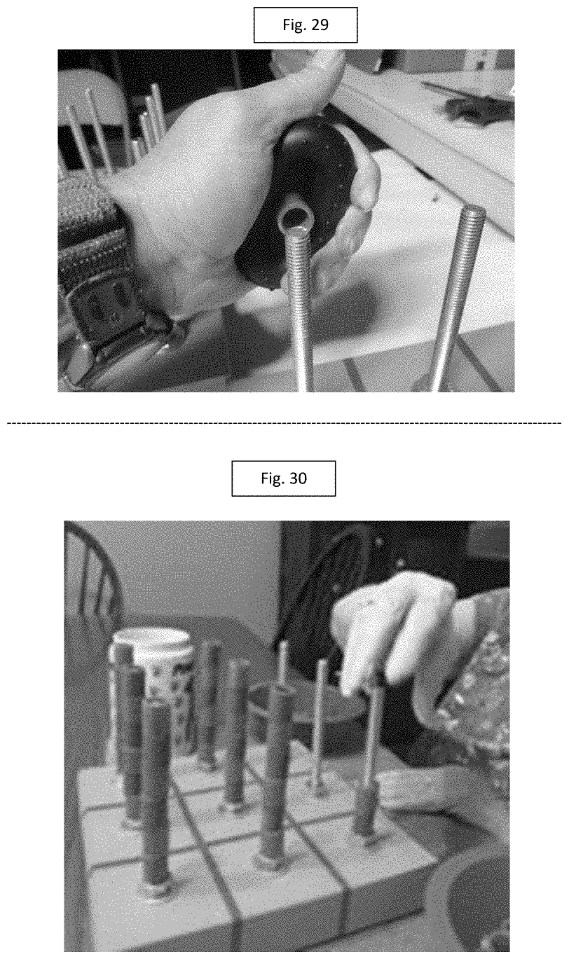

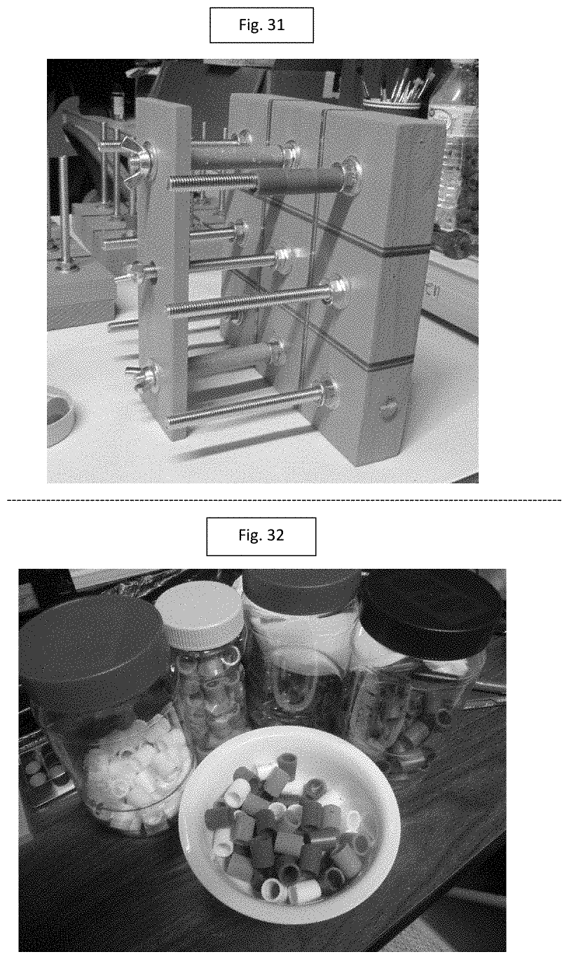

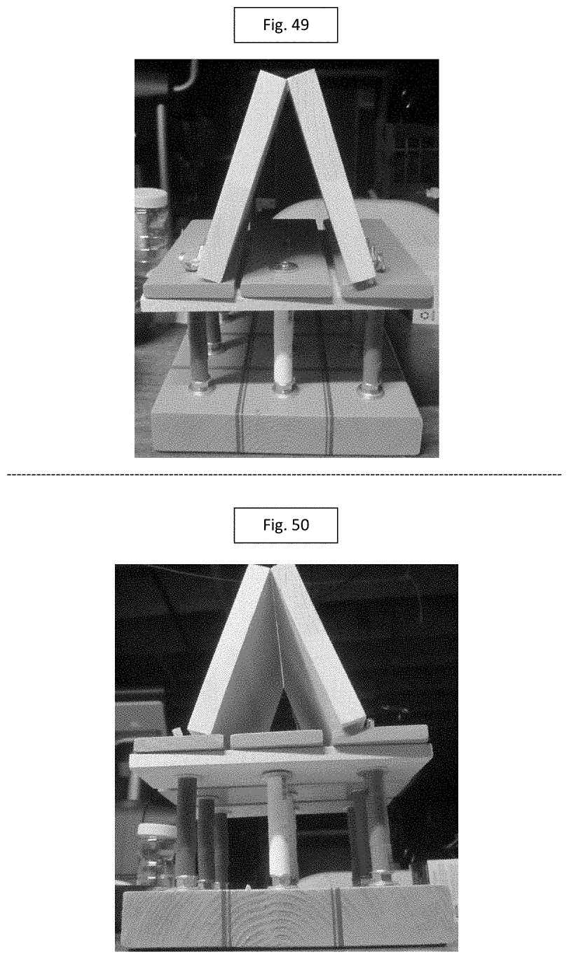

2. comprises of the original 9 inch by 9 inch, Square, 3 bolts by 3 bolts=total of 9 bolts of Stackable 3D Tic Tac Toe. Same as claim #1 but the game/therapy board has the main focus of being a therapy board. The other “Stackable 3D Tic Tac Toe” sizes, shapes, and additional colors of game pieces/tubies are excellent additions to be used for therapy as well. The following text of this claim explains in detail what the additional items that also comprises “Stackable 3D Tic Tac Toe” being used as a therapy board.
To understand more about this main focus of the game also being a therapy board, I believe a review of the letter directly following, from my occupational therapist, Kelly, explains the use well! As the inventor of this game/therapy board, I can tell you that there is a totally different emotion involved and seen, when the game is used as a therapy board. The emotions are not the highs of winning or the lows of losing but of “love of a helpful and healing” therapy! I have seen the therapy board, help and in some ways, encourage healing of my fellow patients who were stroke victims, having injured fingers and hands, handicapped both physically and mentally, etc. When Stackable 3D Tic Tac Toe is used as a therapy board, the main focus is not for the board to produce a winner but to produce therapy actions that yield fine motor coordination, strength, pinch strength and also being a very functional activity. The therapy board has a mental component as stroke victims use the board in challenging procedures and building their decision making process.
Following is a letter from Kelly who was my occupational therapist which explains how she uses “Stackable 3D Tic Tac Toe” as both a therapy board and a game. The letter was converted from a pdf file so there are a couple of small conversion issues that can be seen.
FIG. 10, located in the Drawings file, page 7 shows an exact copy of the endorsement/reference letter showing Kelly's signature.
The letter is not intended to be “bragging” about the game/therapy board but to help recognize the functionality of the game/therapy board and how it achieves its intended and sometimes non-intended purposes and goals.
Sep. 29, 2022, To whom it may concern:
Richard McCracken has developed an awesome “Stackable 3D Tic Tac Toe Board” which can be utilized as a vertical game board or as a functional activity. This board game is unlike anything I have seen in 20 years practicing as an occupational therapist. I have not encountered a board game like this in any rehab catalog or online that offers what this game can.
I had the privilege to work with Richard after his traumatic saw injury to his left hand. He had a tendon repair of his fingers which led him to occupational therapy services for over three months. He mentioned that he made wood working projects and had an idea for me and my patients! During his recovery he was able to fabricate this awesome game.
As a certified hand therapist, I utilize this game on a daily basis to promote fine motor coordination, strength, and pinch strength with my clients. They may at times play tic tac toe against one another or I have them manipulate the pieces and place on the board for a functional activity. It not only can address the physical limitations of the hand, but also adds in a social and mental component as they work on strategizing as well.
I hope you find this as interesting and as dynamic as I have. I think it is a brilliant activity not only for my clients that have a hand injury, but it is diverse enough to encourage cognitive and social impairments as well, offering a side of fun!
Respectfully,
Kelly A. Redding, MOT, OTR/L, CHT
FIG. 26, located in the Drawings file, page 17 shows that when the game is used as a therapy board, items that are used in themselves as therapy tools can become accessories with the game.
FIG. 27, located in the Drawings file, page 18, FIG. 28, located in the Drawings file, page 18, and FIG. 29, located in the Drawings file, page 19 shows that the therapy tools are used to pinch the game pieces, hold the game piece tight, then transport the game piece to the game board and placed over a bolt.
Tweezers can be used as well for the pickup and delivery of the game pieces/Tubies to the game/therapy board bolts.
FIG. 30, located in the Drawings file, page 19, shows my mother-in-law who had a stroke, creating a pattern of alternating colors.
She does one color with one hand and then with her weak left hand, she does the other color.
FIG. 32 located in the Drawings file, page 20 shows where Gram's OT said to put all the colors into one bowl, so she can make decisions in both pulling and placing the game pieces.
To use other muscles in therapy, we used a hotdog grabber as our tweezers for the pickup and delivery of the game pieces/Tubies to the game/therapy board bolts. Then the “decision making” therapy continues as you separate all those game pieces/Tubies back into their individual color container. To add more “decision making” therapy, I would have Grammy count how many game pieces/Tubies we had of each color to see if we had lost any.
The occupational therapists that have seen the game being used as a therapy board, explain that being able to place the game pieces onto the game board at varying angles and positions is very important for a patient's therapy. So, the board can be placed at different heights, so the patient has to reach to the different levels producing the different angles in placing the game pieces.
FIG. 31, located in the Drawings file, page 20, shows how by cutting and adding two, three inch pieces of ½ inch either PEX or PVC tubing, the therapy board can be placed on its side and be very stable.
With the therapy board being on placed its side, the placing of the game pieces now produces many new angles of finger, wrist, and hand motion as the pieces can either be pushed on pulled on from the back side or pushed on from the front.
There is also the focus of the therapy board being used as a “Constructor/Building Unit” with building parts that could be included with “Stackable 3D Tic Tac Toe”. These additional building parts could be made of wood pieces that have 11/16 inch holes drilled in them such as the handle piece but smaller and could be different colors.
FIG. 49, located in the Drawings file, page 30, and FIG. 50, located in the Drawings file, page 30 shows that the additional wood pieces would be used in conjunction with the ⅜ washers and game pieces to build small dollhouse structures looking like cabins.
The therapy patient will have a fun therapy workout in constructing the cabin and then tearing the cabin down for use for the next patient. These prototype structures were just a quick and crude present look of the small cabin structure. Future plans will have the roof wider and cut with the proper angles, so the roof pieces fit correctly.

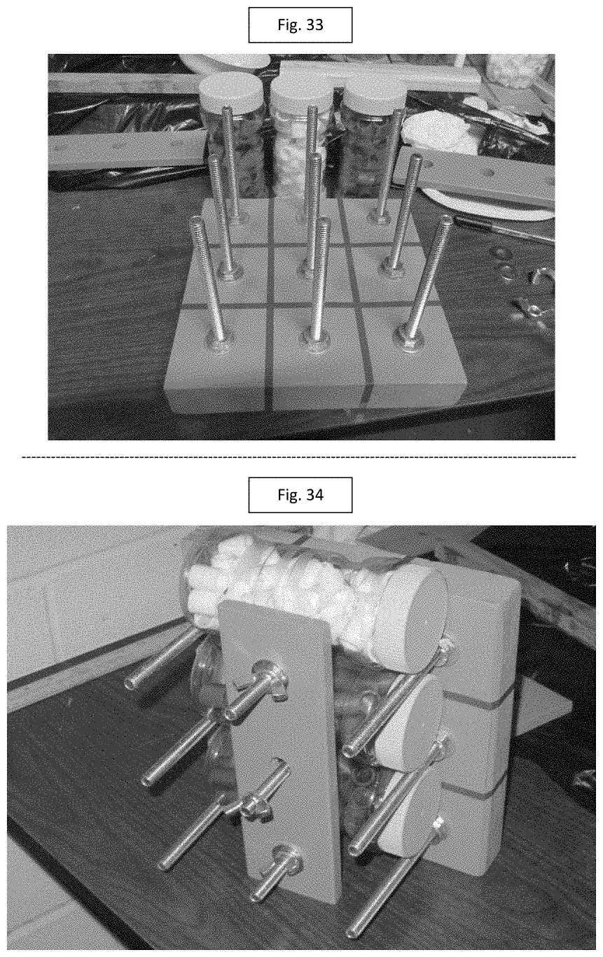

3. comprises of the original 9 inch by 9 inch, Square, 3 bolts by 3 bolts=total of 9 bolts of Stackable 3D Tic Tac Toe board along with the other components in claim #1 but the game/therapy board has the addition of a third player with a third color of game pieces, red, blue, and now white, or equivalent. The following text of this claim explains in detail what the additional items that also comprises “Stackable 3D Tic Tac Toe” being used as a three player game board.
When there are more than two players, “except for partner play-four players”, any player except the player that went first, can go vertical when it is his/her turn. Also, with this same situation with three players playing on an original 3×3 game board, there can be a time when the player that went first will have no choice, as all first level playing positions are already filled with game pieces, but to go vertical first.
FIG. 33 located in the Drawings file, page 21 shows the three player version of Stackable 3D Tic Tac Toe which has been tested and is a lot of fun and it is a very much of a challenge as your mind is called upon to think outside of the regular two player format of Tic Tac Toe.
FIG. 34 located in the Drawings file, page 21 shows that with the addition of a third color, white, the 3^{rd} container holding the white pieces/tubies, can be fitted nicely on the outside ledge and held down by the handle with the tightening of the wingnuts.
The Rules of the Game/Therapy Board
[Regular Play—2 Players] or [Partner Play—4 Players, 2 players (Partners/Team—Red game pieces) and 2 players (Partners/Team—Blue game pieces), same rules just playing as partners.] Three Individual Players—If the game comes with three colors, usually red, white and blue, then 3 individual players can play the game. The same rules are followed with an exception and a possible additional procedure. The exception is that in the regular play rules, the player that went first is not allowed to go vertical until either the 2^{nd} or 3^{rd} player goes vertical, but there can be the case, where the player who went first will have to go vertical first due to no openings on the first level. A possible additional procedure is that once there is a winner, play can continue to see which of the 2 players left, wins 2^{nd} place.
Rules: There is a winner every time! One wins by meeting the actions listed in either of the rules, 1, 2, or 3:
1. Horizontally/Across—having 3 of your color pieces in a row on the same level, (regular Tick-Tac-Toe per level).
2. Vertically/Upward—having 4 of your color pieces touching each other.
3. If a player knocks over any stack of pieces, that player loses the game.
4. The player that goes first cannot place one of his pieces vertically/upward until one of the players that did not go first, does so. When the first game piece is placed on the second level, the second level does not need to be filled by any number of game pieces as the next player could place his game piece in the third level on top of the piece that was just placed in the second level. This pattern of play continues for all levels until there is a win.
Summary: Play continues both horizontally/across and vertically/upward until one of the players wins or loses as described in Rules 1, 2, or 3. There is always a winner of every game! If the game is being played for rehab therapy, the opponent of the injured player, needs to play with lest dominate hand for fairness. Patent Pending!

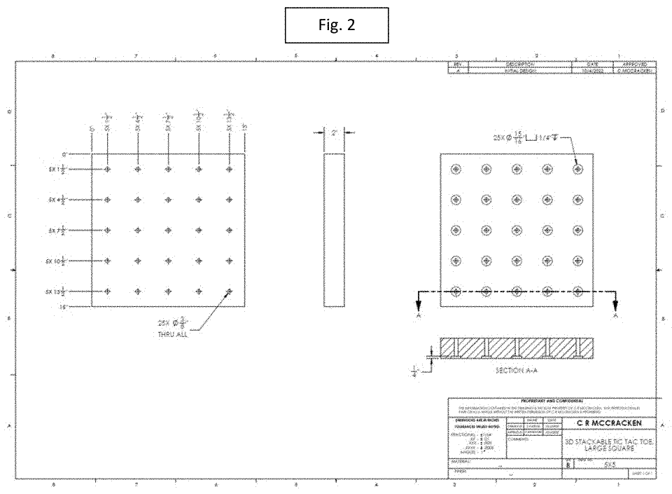

4. FIG. 2 located in the Drawings file, page 2 shows a CAD black and white drawing, where claim #4 comprises of the expansion of the original design covered in claim #1, yielding the “Mind Blowing”, “Mega Board” version of Stackable 3D Tic Tac Toe, 15 inch by 15 inch, Square, 5 bolts by 5 bolts=total of 25 bolts
The following text of this claim explains in detail what the additional items that also comprises “Stackable 3D Tic Tac Toe” being used as a 15 inch by 15 inch, Square, 5 bolts by 5 bolts game board. This claim not only claims the “Mega Board” version of Stackable 3D Tic Tac Toe, 15 inch by 15 inch, Square, 5 bolts by 5 bolts=total of 25 bolts, but any size of square “Mega Game board”, either larger or smaller where the game of “Stackable 3D Tic Tac Toe” can functionally be played as described in this claim #4.
The mega board brings increased challenges with additional players with additional color pieces and additional playing area. Moving from the traditional 3×3 grid of Tic Tac Toe, “blows one's mind”. In playing the game on the “Mega Board”, the best fit is to have 4 or more independent players and colors. If there are only two players playing on the mega board, the rules must be changed to where there must be four of the same color pieces touching horizontally/across, the same as vertically/up and down. With three players, the rules can be switched back to the regular three of the same color pieces touching horizontally/across but you will spend most of your time blocking the player that went first. Four players with four different color pieces, seems to work the best playing on the “Mega Board”. Playing on the “Mega Board” with four players with four different color game pieces makes for a real need for a player to view and focus on the entire large board and all angles of your opponent's moves! In all the games that I played on the “Mega Board”, there was hardly any time where game pieces were placed on levels past the first. Most, if not all of the games, were won on the first level and ended quicker than games played on the regular 3 bolt by 3 bolt game board. So much space, so much fun! The reason that the thickness of the “Mega Board” increased from the thickness of 1.5 inches of the regular 3×3 board to two inches, is that you cannot buy 2 inch by 16 inch plank at Lowes. I went to a nearby Amish sawmill and was able to purchase one inch thick board that was 16 inches wide and 8 feet long.
FIG. 15, located in the Drawings file, page 10 shows where I cut two boards, 15 by 15, alternated the grains, and screwed the boards together, thus making the thickness, two inches which makes the game board strong and heavy.
Warning: Do Not drop the “Mega Board” on one's toes!

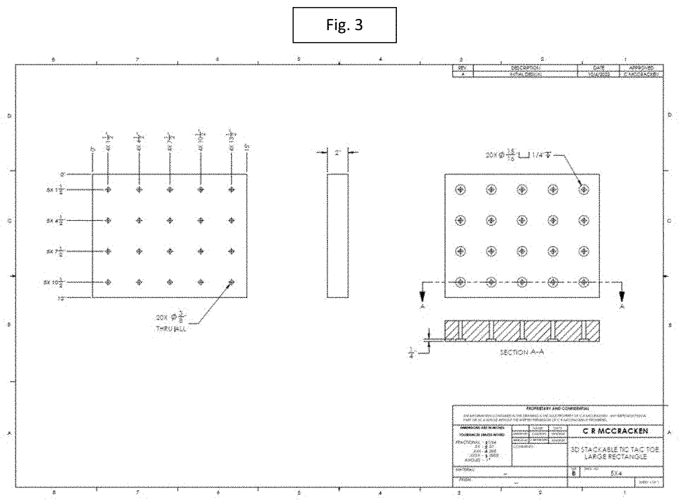

5. comprises of the reduction of the “Mega Board” version of Stackable 3D Tic Tac Toe, 15 inch by 15 inch, Square, listed and explained in claim #4.
FIG. 3, located in the Drawings file, page 3 shows a CAD black and white drawing, the reduction of the “Mega Board” produces “The Rectangular” version of Stackable 3D Tic Tac Toe, 15 inch by 12 inch, Rectangular, 5 bolts by 4 bolts=total of 20 bolts, called the “Semi Maga Board”.
The following text of this claim explains in detail what the additional items that also comprises “Stackable 3D Tic Tac Toe” being used as a 15 inch by 12 inch, Rectangular, 5 bolts by 4 bolts game board. This claim not only claims the “Semi Mega Board” version of Stackable 3D Tic Tac Toe, 15 inch by 12 inch, Rectangular, 5 bolts by 4 bolts, but any size of the rectangular, “Semi Mega Game board”, either larger or smaller where the game of “Stackable 3D Tic Tac Toe” can functionally be played as described in this claim #5.
Even though this version of Stackable 3D Tic Tac Toe has the same rules as the “Maga Board, 5×5”, there is a very important difference. This is the first version that the game board is not a square but a rectangle! This concept of being able to play Stackable 3D Tic Tac Toe on a game board that is not a square game board, leads to many more configurations. These “many more configurations” will be listed throughout the following pages of claim #6. To test and evaluate this rectangular version, the 5×4, the “Maga Board, 5×5”, was used.
FIG. 35, located in the Drawings file, page 22 shows that in converting the square “Maga Board, 5×5” to a playable rectangular board, several rubber bands were placed over one of the outside bolt rows, preventing any game pieces/tubies, to be placed in this row.
The test was positive! Playing Stackable 3D Tic Tac Toe on a rectangular game board with the same rules as the “Mega Board, 5×5” version of Stackable 3D Tic Tac Toe was successful and a lot of fun!

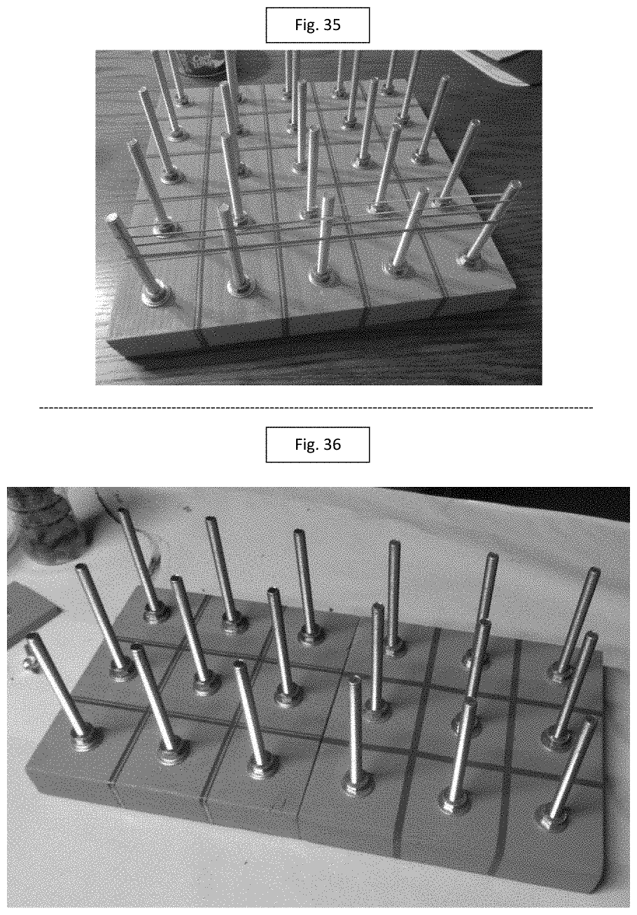

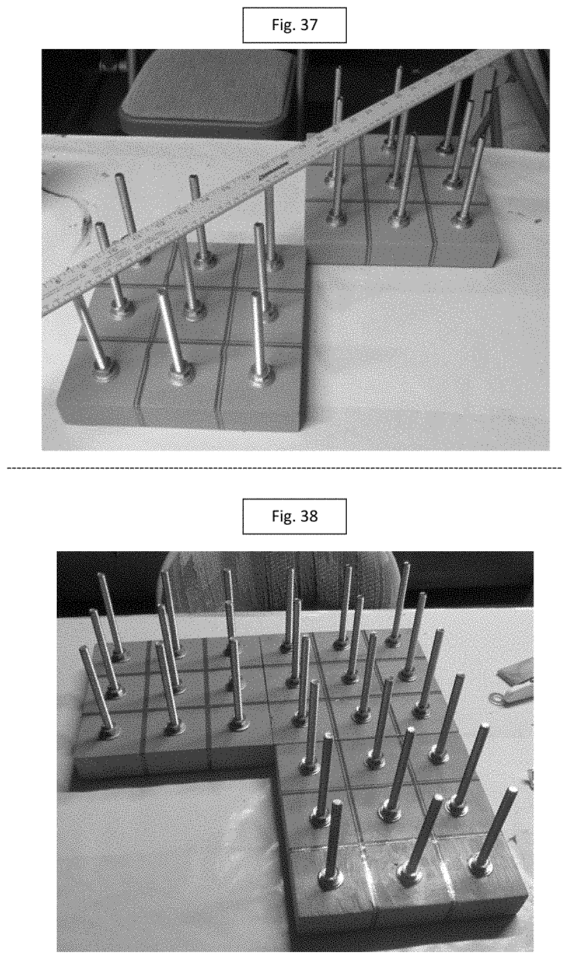

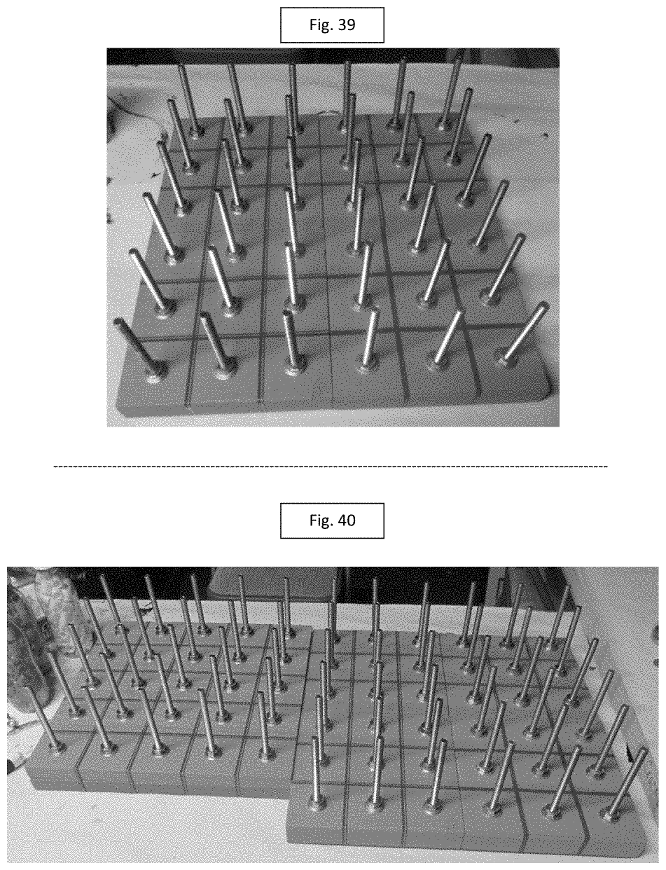

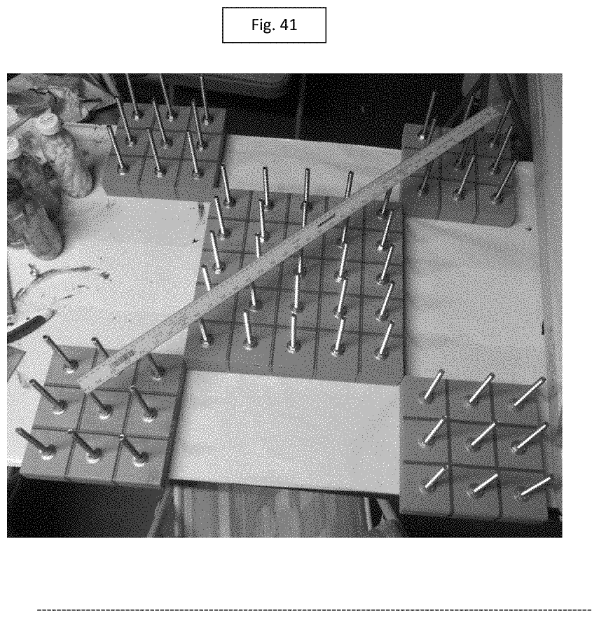

6. This claim collectively comprises all the many collections, configurations, and sizes of the game board or the “entire collective” game board, where Stackable 3D Tic Tac Toe can be successfully played. To define the two types of “game board” and [“entire collective” game board], the following explanation is given. The two types are just different modules of the game board. The phrase “game board” is a single, solid, game board that can be picked up in one piece. For example, the original 3×3 game board and the “Mega Board 5×5” game board.
FIG. 36, located in the Drawings file, page 22, FIG. 37, located in the Drawings file, page 23, FIG. 38 located in the Drawings file, page 23, FIG. 39, located in the Drawings file, page 24, FIG. 40, located in the Drawings file, page 24, and FIG. 41, located in the Drawings file, page 25 all show examples of a [“entire collective” game board] which is where multiple game boards are placed together to produce an entire collective game board on which to play Stackable 3D Tic Tac Toe.
By no means do these six figures show all of the different configurations of the [“entire collective” game board] type. As stated in the opening sentence, “this claim is collectively covering all the many collections, configurations, and sizes of a game board or the “entire collective” game board, where Stackable 3D Tic Tac Toe can be successfully played.”
FIG. 36, located in the Drawings file, page 22, and FIG. 37, located in the Drawings file, page 23, show where two original, 3×3, game boards are placed together to successfully play Stackable 3D Tic Tac Toe.
FIG. 36, located in the Drawings file, page 22, shows the two boards placed side by side yielding a 6×3 [“entire collective” game board].
FIG. 37, located in the Drawings file, page 23, shows the two boards placed in a diagonal configuration yielding one longest line of play of 6 bolts for game pieces where if the two game boards were placed side by side, you would have three longest line of play of 6 bolts for the game pieces.
FIG. 37, located in the Drawings file, page 23 shows where to help in lining up the two game boards when placed diagonally, one can use a straight edge.
FIG. 38, located in the Drawings file, page 23, shows a configuration that can be created when one has three of the original 3×3 game boards, kind of a “L” shaped [“entire collective” game board].
FIG. 39, located in the Drawings file, page 24, shows where four of the original 3×3 game boards are placed together to create a “Huge Mega Board” that is a huge 6×6 [“entire collective” game board].
FIG. 40, located in the Drawings file, page 24, shows a 5×5 “Mega Board” placed side by side with four original 3×3 game boards.
I guess to have a descriptive equation to list this configuration, one could write, [a 11×6—a 1×5]=Configuration of FIG. 40, located in the Drawings file, page 24.
FIG. 41, located in the Drawings file, page 25, shows a “Mega Board”, 5×5, at the center and an original 3×3 game board at each of the four corners of the “Mega Board”, 5×5. This configuration yields two diagonal, “longest line of play”, of eleven bolts.
So, enjoy figuring out all the many options of the different collections and configurations that you can create with how many of the different versions/types of Stackable 3D Tic Tac Toe game boards that you have.

7. In claim #6, the following sentence was listed, “this claim collectively covers all the many configurations and sizes of a game boards or the “entire collective” game boards, where Stackable 3D Tic Tac Toe can be successfully played. Not only are all the configurations and sizes collectively claimed, but also in this claim, #7, all the different materials that can be used to construct the game board and its accessories! For example, the game board could be made of metal of plastic instead of wood. The metal threaded bolts could be replaced by wood or plastic dowels but since you would lose the threads of the bolt, there would be no hold down force, etc. The game pieces/tubies, instead of being made of PEX or PVC plastic could be tubing made from metal or wood rings, etc. The grid lines could be changed from pint strip to being painted, etc.
This claim, #7, also claims that even if the name of the game is changed by an “outside source” and the game still follows previous and later listed claims such as rules, changes in rules and additional rules, listed procedures, structures, different materials, etc., and the substance/DNA matches “Stackable 3D Tic Tac Toe” listed text, it should be as Shakespeare said, “a rose by any other name, is still a rose! To give some examples of different suggested names, “3D Tic Stack Toe” and “3D Tic Tac Tubie”, but still “Stackable 3D Tic Tac Toe”! This claim also lays claim to naming the game pieces as “Tubies” as they are cut from tubing.

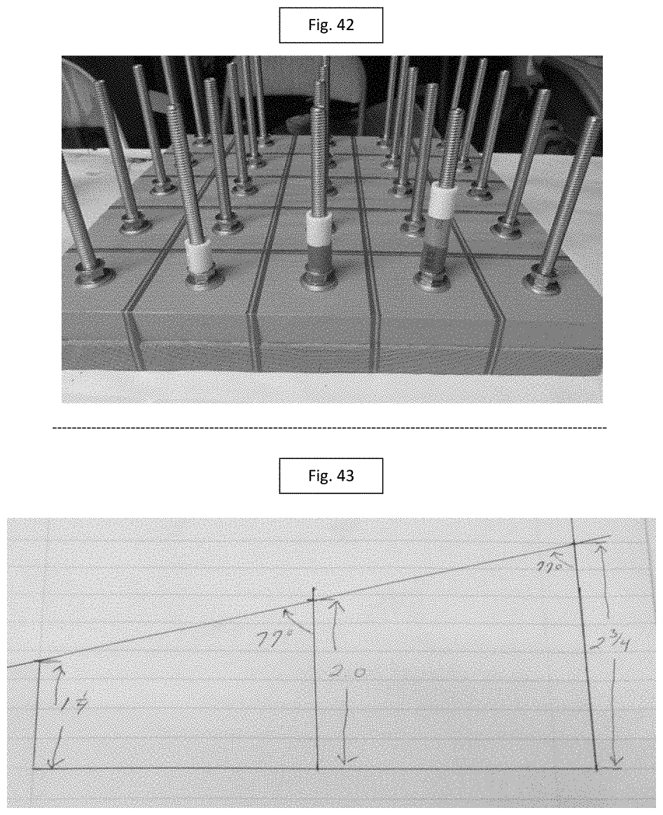

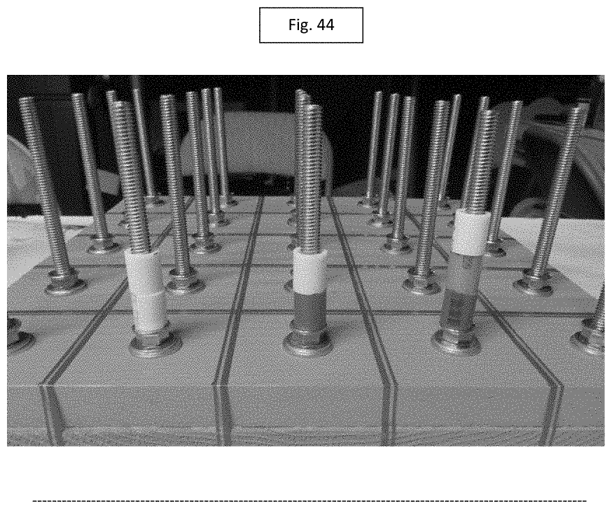

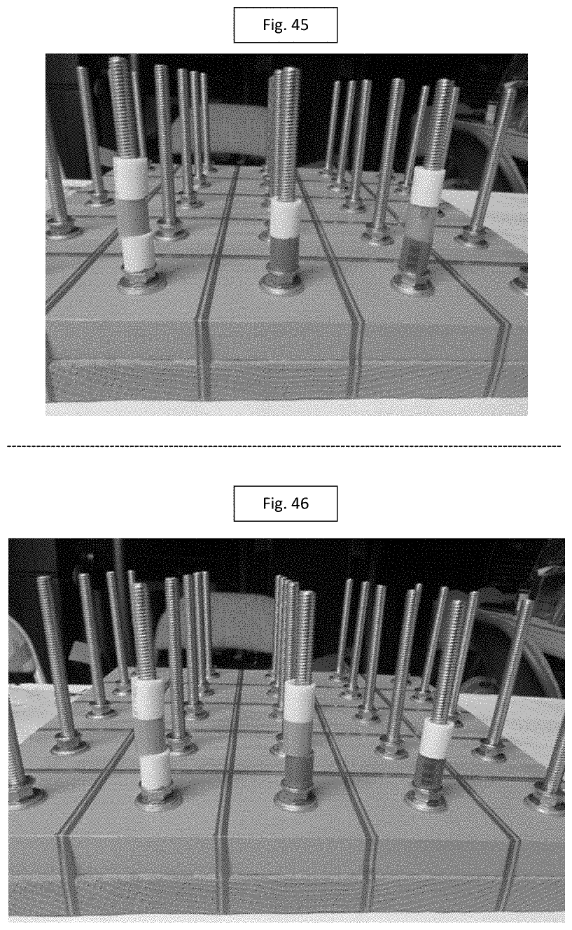

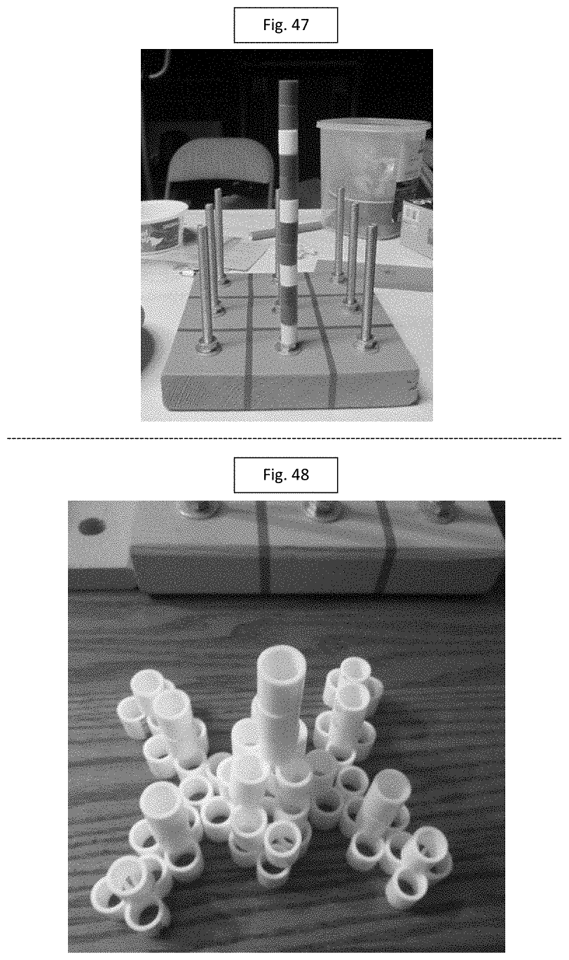

8. This claim comprises the subjects of Additional Rules and a few fun procedures using some of the components in various ways which are listed below:
1. Additional Rules/“House Rules”
2. Computer Programming of “Stackable 3D Tic Tac Toe”
3. The subset of the game in seeing how many game pieces/“Tubies” can be stacked on one bolt before the tower falls.
4. Stacking game pieces/Tubies off the game board for “fun building”.
1. “Additional Rules”/“House Rules: As the inventor of Stackable 3D Tic Tac Toe, I like and enjoy the more simple, easy to learn, not complicated, game rules that have been listed so far for the game. But you know how “gamers” are, those who spend a lot of time playing games, love to add rules to the game, to make the game more variable and more challenging in knowing how to win. Also, these additional/“House Rules” are a way of producing a winner of the game much faster than just following the regular rules. So, here are two rules that have been suggested as the game was evaluated, tested, and played.
(1.) The “Diagonal Rule” is where a win for a player is accomplished by having three pieces of the same color in a diagonal line of 77 degrees, see FIG. 42, located in the Drawings file, page 26 and FIG. 43, located in the Drawings file, page 26.
The calculation of the 77 degree angle of the Diagonal Rule shown in FIG. 43, located in the Drawings file, page 26, was drawn to scale from the “setup” seen in FIG. 42, located in the Drawings file, page 26, which establishes the standard/requirement of the Diagonal Rule.
More simply stated, there will be one more game piece of same color, on the next bolt, over three bolts, either inclining or declining. Not sure if that was more simply stated?
(2.) The “Uneven, Top Three, Same Color of Piece Rule”, I know, what a long and complicated name of the rule but the name of the rule does need to be descriptive of how the rule works.
FIG. 44, located in the Drawings file, page 27, FIG. 45, located in the Drawings file, page 28, and FIG. 46, located in the Drawings file, page 28, are all examples of a win for the white game piece using the “Uneven, Top Three, Same Color of Piece Rule”.
I have a few negatives with this rule. First, it is in direct opposition to one of basic rules of the game that states the following: 1. Horizontally/Across—having 3 of your color pieces in a row on the same level, (regular Tick-Tac-Toe per level).
Second, now this rule allows for a win when the three game pieces of same color occupy the top of the stack for three consecutive stacks, no matter the difference of heights of the three stacks! I think this rule is stretching things, I mean stretching stacks! I also think this rule allows for a “sneaky” win! I do not have any negatives with the “Diagonal Rule” as it “clean cut” but as you look at the boundaries and requirements of the “Uneven, Top Three, Same Color of Piece, Rule”, the “Diagonal Rule” is actually a subset of the “Uneven, Top Three, Same Color of Piece, Rule”.
2. This claim, #8, also concerns all electronic programming of all versions of the Stackable 3D Tic Tac Toe game. As the game was played, there were some players who were thinking about and wanted to accomplish computer game programming of the game. It seemed to these gamers that the electronic programming of the game was feasible. Knowing a little about computer programing, I asked the question as to how do you program a hand and finger skill, the “keep on stacking game pieces until a tower of game pieces is knocked over to lose”? The gamers answer was that you would have to set a high limit of the height of game pieces. I lay claim to those who program the functions of the game, Stackable 3D Tic Tac Toe, to create an electronic game, the programmers need to pay an “inventor's fee” to do so.
3. This claim, #8, also concerns this particular use of the game/therapy board, the focus is on the action word of “Stackable” in the game title of Stackable 3D Tic Tac Toe. The regular game rules can be dropped, and the game board and game pieces can be put into “a lot of fun action” by simply stacking pieces on a single bolt, to see how high the game pieces can be stacked. The record stood at 17 for a long time, then a player broke the record on May 23, 2022, stacking 20 game pieces and then a couple of tries later, the record that still stands at 21, was accomplished. I think that player should become a surgeon, having hands that are steady, agile, and skillful!
FIG. 47, located in the Drawings file, page 29, shows my attempt at stacking which proved that I am not a record contender and should not be a surgeon! FIG. 47, located in the Drawings file, page 29, shows my stack at twelve high. I did make it to 14 but as I was stacking my 15^{th} game piece, I experienced my downfall!
4. In talking about stacking, you do not have to accomplish your stacking on one of the bolts, while you are waiting your turn or whenever or wherever, you can stack games pieces/“tubies” for challenge and fun, see FIG. 48, located in the Drawings file, page 29.

9. comprises of the expansion and reduction of the sizes of the “Stackable 3D Tic Tac Toe” game board. In the expansion of the original design covered in claim #1, brings one to be looking at games such as chess where the table size board game expands to a court size board where players pickup one chess piece with both arms and walks the piece to the acceptable square, Stackable 3D Tic Tac Toe can follow the same route, to a court size board and even to a large field size playing area. You could have poles in the ground in a Tick-Tac-Toe grid, the game pieces would be lightweight but large. The two teams could have numerous players but the same number of players on each team. The basic rules of Stackable 3D Tic Tac Toe would be adjusted and adapted to teams playing on the much larger playing area. The team players would get a great athletic workout in getting the game pieces up over the poles by using ladders, human ladder chains, or ropes or established ladders at each pole. This could be a great strength training and climbing game for the military and police and could even become a college and Olympic sport! Adding clock timing such as using a clock in chess matches is an option to further add to the strategies of playing Stackable-3D-Tick-Tac-Toe wherever and however it is played.
This claim, #9, also claims the reduction sizes of game boards where Stackable 3D Tic Tac Toe can be successfully played, which describes and explains the smaller or micro sizes of the game. With the bolts sticking up, allowing the game pieces to stay in place and to be held from sliding off the board, even when traveling, is definitely a great advantage of the design. The size of the game board, the game pieces and other accessory parts could be made much smaller and have a small “footprint”. Small enough to be on the side of any restaurant table and be pulled into play while waiting for your food. Small enough to be played in cars, buses, ships, airplanes, etc. Small enough to be pulled from a pocket or a purse. Stackable 3D Tic Tac Toe could be transported and played, just about everywhere.
